(19) United States
(12) Patent Application Publication
(10) Pub. No.: US 2004/0042986 A1
(43) Pub. Date: Mar. 4, 2004

(54) Method of preventing or alleviating prickly heat using synthetic mild personal wash cleanser

(75) Inventors: Frank Meyer, Fort Lee, NJ (US); Carol Kregler Vincent, Wanaque, NJ (US); William Dwight Becker, Rockaway, NJ (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/234,269

(22) Filed: Sep. 4, 2002

(51) Int. Cl. A61K 7/06

(52) U.S. Cl. 424/70.1

(57) ABSTRACT

The present invention relates to applicants unexpected observation that there is a link between specific pH ranges and the onset and/or exacerbation of prickly heat. By using cleanser with defined pH range, the prickly heat can be prevented and/or alleviated.

Applicants have also observed that prickly heat is associated with blocked pores. In a second embodiment, the invention provides a method of increasing number of unblocked pores using cleanser within same defined pH range.

### FIELD OF THE INVENTION

The present invention relates to methods for treating a skin condition known as miliaria rubra (commonly referred to as “prickly heat”).

### BACKGROUND OF THE INVENTION

Consumers who use personal wash cleanser products (e.g., personal wash liquids and bars) are, of course, interested in products which cleanse and kill germs. There are, however, many additional body and skin concerns which are important to consumers, and which consumers do not necessarily associate as conditions which can be addressed using personal wash cleanser system. Among these, for example, are sweaty skin, itchy skin, prickly heat, greasy or oily skin, sensitive skin and rashes or irritations of the skin.

Since the 1950's, it has been known that excessive washing with conventional soap could cause prickly heat or its characteristic plugs (see J. P. O'Brien, III, “The Pathologic Effect of Excessive Soaping on the Pores of the Skin”, J. Invest. Derm, pp 134-140 (15), 1950). At this point in time, people were speculating about effects like lipid depletion being responsible for prickly heat. Since soaps were known to have high lipid depletion potential, it was concluded that washing with soap would at least exacerbate the condition (i.e., on the basis of lipid depletion).

Unexpectedly, applicants have now found that there is a link between the pH of a cleanser and onset of prickly heat. Specifically, if cleanser products are kept within critical pH ranges, there is a strong link between the pH and deterring onset of prickly heat. Accordingly, use of cleansers within the delivered ranges can alleviate or prevent this prickly heat skin condition.

Miliaria rubra, commonly referred to as prickly heat, is an inflammatory disorder of human skin which is characterized by multiple small lesions at the site of sweat pores on the skin. It is believed that the condition is brought about by blockage of sweat ducts and the resulting escape of sweat into the epidermis. The blockage of the sweat duct is in turn caused by accumulation of a bacterial substance known as EPS (“extracellular polysaccharide substance”). See Mowad et al. “The Role of Extracellular Polysaccharide Substance Produced by Staphylococcus Epidermidis in Miliaria”; J American Academy Dermatology, pages 729-733 (33), 1995.

As far as applicants are aware, the link between specific pH range (e.g., greater than about 4.0 to about 7.5) and the result that greater number of sweat ducts or glands remain unblocked (therefore alleviating the magnitude or deterring the onset of prickly heat) is unknown.

U.S. Pat. No. 5,362,488 to Sibley et al. discloses a buffered diaper rash cream wherein the buffering system results in the cream having a preferred pH in the range of about 4.5 to 6.0, most preferably about 5.2. The pH is said to neutralize acidic and basic by-products of urine and fecal matter and therefore prevent diaper rash.

Although there is some overlap of the pH ranges of the cleaner products of the subject invention, there is no teaching or suggestion of such cream being used effectively against prickly heat condition.

### BRIEF DESCRIPTION OF THE INVENTION

The present invention relates to a method of alleviating the effect of, or deterring the onset of prickly heat by using personal wash cleanser products (liquids, creams, bars, etc.) having pH of greater than about 4.0 to less than about 7.5.

When the pH is too alkaline, this is believed to create an environment in which bacteria associated with the onset of prickly heat may flourish and subsequent irritation and blocking of sweat glands may occur. A more acidic environment (e.g., pH below about 7.5) is believed to control the bacteria (e.g., make them less EPS producing) and cause beneficial effects noted above. On the other hand, if pH is too low, this appears to cause significant irritation of the skin which also then appears to be associated with blockage of the sweat glands (by narrowing of the sweat duct due to irritation response). This then in turn leads to an environment where prickly heat may flourish.

In a second aspect of the invention, therefore, the invention also relates to a method of increasing the number of unblocked sweat glands in a given area of skin by utilizing personal wash cleanser compositions having pH of greater than about 4.0 to less than about 7.5.

### BRIEF DESCRIPTION OF THE FIGURES

In order to create Miliaria Rubra symptoms under test conditions, a skin occlusion model was used (see Holzle and Kligman, “The Pathogenesis of Miliaria Rubra: Role of the Resident Microflora”, J. Dermatology, pp 117-137 (99) 1978, a copy of which is hereby incorporated by reference into the subject application). Skin was occluded using plastic Hill-Top® chambers and acidic oil-in-water emulsion (pH 4.5) served as test product. Untreated, but equally occluded side served as control. As seen in FIG. 1, acidic treatment of the skin caused less sweat gland blockage than no treatment for up to 48 hours.

FIG. 2 shows that, as potential treatments become more acidic (but still above 4.0), the number of unobstructed glands increases. This correlates with improvement of skin condition.

FIG. 3 shows that under occlusive conditions, soap produces 100% blockage of the sweat glands in the area treated. The syndet product, however, kept a significant number of sweat glands unobstructed. FIG. 3 also shows that, as the bacteria level on the skin remains lower, the number of unobstructed glands is generally higher.

FIG. 4 shows that the same as FIG. 3 is true when evaluating a series of marketed soap and non-soap based products. The soaps always cause a complete blocking of the sweat glands under occlusive conditions. In contrast, the syndets show significantly less blockage, with the more neutral syndets causing the least blockage. At lower pHs (but still within treatment window) there is slightly more blockage, presumably due to irritation effect.

FIG. 5 shows the results of in-vitro EPS production test (see Pfaller et al. “Development of the quantitative Micro-Test for Slime Production by Coagulase-Negative Staphylococci” Eur. J. Clin Microbiol. Infect. Dis., pp 30-33 (7), 1988, a copy of which is hereby incorporated by reference into the subject application). Staphylococcus epidermidis, the species thought to cause miliaria rubra, was grown in test tubes containing Trypticase Soy Borth (TSB) under various pH conditions. As can be seen, the optimum EPS production (and therefore potential for sweat gland blockage) was reached at pH about 6.5 to 7.5. Such skin pH values are easily obtained by exposing normally acidic skin to alkaline cleansers like conventional soap.

FIG. 6 shows the effects of washing with conventional soap compared to washing with non-alkaline syndet products (one bar, one liquid). As seen from the figure, the non-soap products maintain a more acidic skin pH, whereas the soap pushes skin pH to about 7, which in turn is the optimum area for EPS production and where gland blocking will occur. Thus, it can be seen that non-alkaline syndet (which allow to skin pH stay lower) are more beneficial relative to conventional soaps.

FIG. 7 shows that least blockage occurs when there is a combination of both lower pH and mildness (upper right quadrant) and conversely, most blockage is found where there is a combination of higher pH and less mildness (lower left).

### DETAILED DESCRIPTION OF THE INVENTION

The present invention relates to method for treating the skin condition commonly referred to as “prickly heat”. In particular, the invention relates to a method for alleviating the severity or deterring the onset of prickly heat by using personal wash cleansers (whether in the form of a bar, liquid, cream etc.) having a pH range from above about 4.0 to less than about 7.5.

At product pH ranges above about 7.5 (too alkaline), while not wishing to be bound by theory, it is believed the resulting skin environment allows bacteria to proliferate which leads to inflammation and blocking of the sweat glands and thereby causing prickly heat. However, if pH is too acidic (below 4.0), the irritation caused by the acidic environment also may lead to blocking of sweat glands. The key is to find a critical balance between sufficiently low pH so that bacteria will not flourish but not so low as to cause irritation.

In a second embodiment of the invention, the invention relate to a method of decreasing the number of blocked sweat glands (or conversely increasing the number of unblocked sweat glands) by using personal wash cleansers having pH of above about 4.0 to less than about 7.5.

The effect seen in the invention is a combined pH and mildness effect (the most mild possible without raising pH so high that bacteria will begin to flourish) and is truly not dependent at all on the particular composition used other than it must fall within the defined pH window.

Thus, the cleanser may be in bar, liquid, cream, emulsion or any other form suitable for application to the skin (including deposition from nonwoven substrate).

If a bar composition is used, typically the bar will comprise 1% to about 80%, preferably 10% to about 75% of a surfactant selected from the group consisting of anionic surfactants (including soap), nonionic surfactants, amphoteric/zwitterionic surfactants, cationic surfactants and mixtures thereof.

Because the pH of the bar cannot be excessively alkaline, the bars are generally not pure soap based bars (where pH tends to be above 7.5), but generally will be bars where synthetic surfactant comprises about 10% to 90%, preferably 20% to 80% of the composition and soap, if present, will comprise 0 to 10% of the composition. These bars will have pH within ranges required by the invention.

Anionic surfactant, other than soap may be an aliphatic sulfonate, such as a primary alkane (e.g., C₈—C₂₂) sulfonate, primary alkane (e.g., C₈—C₂₂) disulfonate, C₈—C₂₂ alkene sulfonate, C₈—C₂₂ hydroxyalkane sulfonate or alkyl glyceryl ether sulfonate (AGS); or an aromatic sulfonate such as alkyl benzene sulfonate.

The anionic may also be an alkyl sulfate (e.g., C₁₂—C₁₈ alkyl sulfate) or alkyl ether sulfate (including alkyl glyceryl ether sulfates). Among the alkyl ether sulfates are those having the formula:

RO(CH₂CH₂O)ₙSO₃M

wherein R is an alkyl or alkenyl having 8 to 18 carbons, preferably 12 to 18 carbons, n has an average value of greater than 1.0, preferably between 2 and 3; and M is a solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆—C₂₂ sulfosuccinates); alkyl and acyl taurates, alkyl and acyl sarcosinates, sulfoacetates, C₈—C₂₂ alkyl phosphates and phosphates, alkyl phosphate esters and alkoxyl alkyl phosphate esters, acyl lactates, C₈—C₂₂ monoalkyl succinates and maleates, sulphoacetates, and acyl isethionates.

Another class of anionics are carboxylates such as follows:

R—(CH₂CH₂O)ₙCO₂M

wherein R is C₈ to C₂₀ alkyl; n is 0 to 20; and M is as defined above.

Another carboxylate which can be used is amido alkyl polypeptide carboxylates such as, for example, Monteine LCQ^{(R)} by Seppic.

Another surfactant which may be used are the C₈—C₁₈ acyl isethionates. These esters are prepared by reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

Isethionate may comprise 0 to 60% of the total composition.

Zwitterionic surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. A general formula for these compounds is:

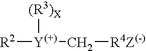

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Amphoteric detergents which may be used in this invention include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:

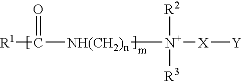

where R¹ is alkyl or alkenyl of 7 to 18 carbon atoms;

R² and R³ are each independently alkyl, hydroxyalkyl or carboxyalkyl of 1 to 3 carbon atoms;

n is 2 to 4;

m is 0 to 1;

X is alkylene of 1 to 3 carbon atoms optionally substituted with hydroxyl, and

Y is —CO₂— or —SO₃—

Suitable amphoteric detergents within the above general formula include simple betaines of formula:

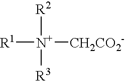

and amido betaines of formula:

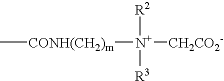

where m is 2 or 3.

In both formulae R¹, R² and R³ are as defined previously. R¹ may in particular be a mixture of C₁₂ and C₁₄ alkyl groups derived from coconut so that at least half, preferably at least three quarters of the groups R¹ have 10 to 14 carbon atoms. R² and R³ are preferably methyl.

A further possibility is that the amphoteric detergent is a sulphobetaine.

The amphoteric/zwitterionic generally comprises 0.1 to 20% by weight, preferably 0.5% to 15%, more preferably 1.0 to 10% by wt. of the composition.

In addition to one or more anionic and amphoteric and/or zwitterionic, the surfactant system may optionally comprise a nonionic surfactant.

The nonionic which may be used includes in particular the reaction products of compounds having a hydrophobic group and a reactive hydrogen atom, for example aliphatic alcohols, acids, amides or alkyl phenols with alkylene oxides, especially ethylene oxide either alone or with propylene oxide. Specific nonionic detergent compounds are alkyl (C₆—C₂₂) phenols-ethylene oxide condensates, the condensation products of aliphatic (C₈—C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxides.

The nonionic may also be a sugar amide, such as a polysaccharide amide. Specifically, the surfactant may be one of the lactobionamides described in U.S. Pat. No. 5,389,279 to Au et al. which is hereby incorporated by reference or it may be one of the sugar amides described in U.S. Pat. No. 5,009,814 to Kelkenberg, hereby incorporated into the subject application by reference.

Other surfactants which may be used are described in U.S. Pat. No. 3,723,325 to Parran Jr. and alkyl polysaccharide nonionic surfactants as disclosed in U.S. Pat. No. 4,565,647 to Llenado, both of which are also incorporated into the subject application by reference.

Preferred alkyl polysaccharides are alkylpolyglycosides of the formula

R²O(CₙH₂ₙO)ₜ(glyCosyl)ₓ

wherein R² is selected from the group consisting of alkyl, alkylphenyl, hydroxyalkyl, hydroxyalkylphenyl, and mixtures thereof in which alkyl groups contain from about 10 to about 18, preferably from about 12 to about 14, carbon atoms; n is 0 to 3, preferably 2; t is from 0 to about 10, preferably 0; and x is from 1.3 to about 10, preferably from 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4- and/or 6-position, preferably predominantly the 2-position.

The nonionic surfactant can also be a water soluble polymer chemically modified with hydrophobic moiety or moieties. For example, EO-PO block copolymer, hydrophobically modified PEG such as POE(200)-glyceryl-stearate can be included in the formulations claimed by the subject invention.

Nonionic generally comprises 0 to 10% by wt. of the composition.

Also included are cationic surfactants. Nonlimiting examples of cationics include stearyldimethylbenzyl ammonium chloride; dodecyltrimethylammonium chloride; nonylbenzylethyldimethyl ammonium nitrate; tetradecylpyridinium bromide; cetylpyridinium chloride; laurylpyridinium chloride; laurylisoquinolium bromide; ditallow(hydrogenated) dimethyl ammonium chloride; dilauryl dimethyl ammonium chloride; and stearalkonium chloride.

In addition, the compositions of the invention may include optional ingredients as follows:

Organic solvents, such as ethanol; auxiliary thickeners, such as carboxymethylcellulose, magnesium aluminum silicate, hydroxyethylcellulose, methylcellulose, carbopols, glucamides, or Antil^{(R)} from Rhone Poulenc; perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer); all of which are useful in enhancing the appearance or cosmetic properties of the product.

The compositions may further comprise antimicrobials such as 2-hydroxy-4,2′4′trichlorodiphenylether (DP300) and/or trichlorocarbon (TCC); preservatives such as dimethyloldimethylhydantoin (Glydant XL 1000), parabens, sorbic acid etc.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) may be used advantageously in amounts of about 0.01% or higher if appropriate.

Cationic conditioners which may be used include Quatrisoft LM-200 Polyquaternium-24, Merquat®-polymer; and Jaguar^{(R)} type conditioners from Rhone-Poulenc; and Salcare®-type conditioners from Allied Colloids.

Polyethylene glycols which may be used include:

| | | |
|---|---|---|
| Polyox | WSR-205 | PEG 14M, |
| Polyox | WSR-N-60K | PEG 45M, or |
| Polyox | WSR-N-750 | PEG 7M. |

PEG with molecular weight ranging from 300 to 10,000 Dalton, such as those marketed under the tradename of CARBOWAX SENTRY^{(R)} by Union Carbide.

Another ingredient which may be included are exfoliants such as polyoxyethylene beads, walnut shells and apricot seeds

The structurant of the invention can be a water soluble or water insoluble structurant.

Water soluble structurants include moderately high molecular weight polyalkylene oxides of appropriate melting point (e.g., 40°to 100° C., preferably 50°to 90° C.) and in particular polyethylene glycols or mixtures thereof.

Water insoluble structurants also have a melting point in the range 40-100° C., more preferably at least 50° C., notably 50° C. to 90° C. Suitable materials which are particularly envisaged are fatty acids, particularly those having a carbon chain of 12 to 24 carbon atoms. Examples are lauric, myristic, palmitic, stark, arachidic and behenic acids and mixtures thereof. Sources of these fatty acids are coconut, topped coconut, palm, palm kernel, babassu and tallow fatty acids and partially or fully hardened fatty acids or distilled fatty acids. Other suitable water insoluble structurants include alkanols of 8 to 20 carbon atoms, particularly cetyl alcohol. These materials generally have a water solubility of less than 5 g/litre at 20° C.

Soaps (e.g., sodium stearate) can also be used at levels of about 1% to 10%. the soaps may be added neat or made in situ by adding a base, e.g., NaOH, to convert free fatty acids.

The relative proportions of the water soluble structurants and water insoluble structurants govern the rate at which the bar wears during use. The presence of the water-insoluble structurant tends to delay dissolution of the bar when exposed to water during use and hence retard the rate of wear.

Another optional ingredient is oil/emollient which may be added as a benefit agent to the bars compositions.

The composition may also be in the form of a liquid.

Generally, such compositions comprise 5% to 60%, preferably 10% to 50% of surfactants wherein surfactants can be any of the surfactants mentioned above and mixtures thereof.

In a preferred system, the anionic is acyl isethionate and amphoteric is betaine such as cocoamidoalkylbetaine.

Such personal wash compositions may optionally include structurant. Suitable structuring materials include swelling clays, for example laponite; fatty acid and derivatives thereof, in particular, fatty acid monoglyceride polyglycol ethers; cross-linked polyacrylates such as Carbopol (TM) (polymers available from Goodrich); acrylates and copolymers thereof; polyvinylpyrrolidone and copolymers thereof; polyethyleneimines; salts such as sodium chloride and ammonium sulphate; sucrose esters; gellants; and mixtures thereof.

Of the clays, particularly preferred are synthetic hectorite (laponite) clay used in conjunction with an electrolyte salt capable of causing the clay to thicken. Suitable electrolytes include alkali and alkaline earth salts such as halides, ammonium salts and sulphates.

The composition may also comprise internal lamellar phase-inducing structurants. Such structurants include C₈—C₂₄ unsaturated and/or branched liquid fatty acid or esters thereof; C₈—C₂₄ unsaturated and/or branched liquid alcohol or ether thereof; and/or C₅ to C₉ fatty acids wherein those structuring have MP below 25° C.

When present, structurants may comprise 0.1 to 25%, preferably 1 to 15% of composition.

The personal wash formulations may also comprise a thickening (or thinning) agent, i.e., a material which maintains the viscosity of this phase as the shear rate thereof is increased during use. Suitable materials include cross-linked polyacrylates such as Carbopol (TM) (polymers available from Goodrich); natural gums including alginates, guar, xanthan and polysaccharide derivatives including carboxy methyl cellulose and hydroxypropyl guar; propylene glycols and propylene glycol oleates; salts such as sodium chloride and the ammonium sulphate; glycerol tallowates; and mixtures thereof.

These agents may comprise 1% to 15% by wt. of the composition.

Other typical components of such compositions include opacifiers, preferably 0.2 to 2.0 wt. %; preservatives, preferably 0.2 to 2.0 wt. %; and perfumes, preferably 0.5 to 2.0 wt. %. Cationic polymers such as Jaguar^{(R)} from Rhone Poulenc and Polymer JR^{(R)} from Amerchol may also be included.

The base compositions may further comprise additional oil/emollient particles (particularly when in lamellar phase) wherein the additional benefit agent may be as set forth below:

Vegetable oils: Arachis oil, cannola oil, castor oil, cocoa butter, coconut oil, corn oil, cotton seed oil, olive oil, palm kernel oil, rapeseed oil, safflower seed oil, sesame seed oil and soybean oil.

Esters: Butyl myristate, cetyl palmitate, decyloleate, glyceryl laurate, glyceryl ricinoleate, glyceryl stearate, glyceryl isostearate, hexyl laurate, isobutyl palmitate, isocetyl stearate, isopropyl isostearate, isopropyl laurate, isopropyl linoleate, isopropyl myristate, isopropyl palmitate, isopropyl stearate, propylene glycol monolaurate, propylene glycol ricinoleate, propylene glycol stearate, and propylene glycol isostearate.

Animal Fats: Acytylatelte lanolin alcohols, lanolin, lard, mink oil and tallow.

Fatty acids and alcohols: Behenic acid, palmitic acid, stearic acid, behenyl alcohol, cetyl alcohol, eicosanyl alcohol and isocetyl alcohol.

Other examples of oil/emollients include mineral oil, petrolatum, silicone oil such as dimethyl polysiloxane, lauryl and myristyl lactate. The above-noted list also applies to emollients which can be used in bars.

Additional emollient/oil generally will comprise, if present, 1% to 20% of the composition.

Other ingredients which may be found in such personal care compositions are as follows:

Organic solvents, such as ethanol; auxiliary thickeners, such as carboxymethylcellulose, magnesium aluminum silicate, hydroxyethylcellulose, methylcellulose, carbopols, glucamides, or Antil^{(R)} from Rhone Poulenc; perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer); all of which are useful in enhancing the appearance or cosmetic properties of the product.

The compositions may further comprise antimicrobials such as 2-hydroxy-4,2′4′trichlorodiphenylether (DP300) and/or Triclorocarbon (TCC); preservatives such as dimethyloldimethylhydantoin (Glydant XL1000), parabens, sorbic acid etc.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) may be used advantageously in amounts of about 0.01% or higher if appropriate.

Cationic conditioners which may be used include Quatrisoft LM-200 Polyquaternium-24, Merquat Plus 3330-Polyquaternium 39; and Jaguar^{(R)} type conditioners.

Polyethylene glycols which may be used include:

| | | |
|---|---|---|
| Polyox | WSR-205 | PEG 14M, |
| Polyox | WSR-N-60K | PEG 45M, or |
| Polyox | WSR-N-750 | PEG 7M. |

Other thickeners which may be used include Amerchol Polymer HM 1500 (Nonoxynyl Hydroethyl Cellulose); Glucam DOE 120 (PEG 120 Methyl Glucose Dioleate); Rewoderm^{(R)} (PEG modified glyceryl cocoate, palmate or tallowate) from Rewo Chemicals; Antil^{(R)} 141 (from Goldschmidt).

Another optional ingredient which may be added are the deflocculating polymers such as are taught in U.S. Pat. No. 5,147,576 to Montague, hereby incorporated by reference.

Another ingredient which may be included are exfoliants such as polyoxyethylene beads, walnut sheets and apricot seeds.

As long as within pH limits defined, the compositions may also be cosmetic “leave-on” compositions as opposed to typical rinse-off products like soaps or shower gels. These compositions (creams, lotions) generally will contain less surfactant (i.e., 0-30%, preferably 1-15% by wt.) but include more ingredients characteristic of cosmetic or commercially acceptable vehicle. For example, the benefit agent composition will comprise 1 to 25% by wt. of the total composition, surfactant may comprise 0.5 to 30% by wt. of the composition and balance will be cosmetic vehicle composition.

The cosmetic vehicle composition (comprising 1% to 99% of total cosmetic, preferably 1-80% of total cosmetic) may comprise an oil or oily material, together with an emulsifier, to provide either a water-in-oil emulsion or an oil-in-water emulsion, depending largely on the average hydrophilic-lipophilic balance (HLB) of the emulsifier employed.

Various types of active ingredients may be present in cosmetic vehicle compositions of the present invention. Actives are defined as skin or hair benefit agents other than emollients and other than ingredients that merely improve the physical characteristics of the composition. Although not limited to this category, general examples include sunscreens, tanning agents.

Typically actives will comprise 1% to 30% of the total cosmetic composition.

Another preferred optional ingredient is selected from essential fatty acids (EFAs), i.e., those fatty acids which are essential for the plasma membrane formation of all cells, in keratinocytes EFA deficiency makes cells hyperproliferative. Supplementation of EFA corrects this. EFAs also enhance lipid biosynthesis of epidermis and provide lipids for the barrier formation of the epidermis. The essential fatty acids are preferably chosen from linoleic acid, γ-linolenic acid, homo-γ-linolenic acid, columbine acid, eicosa-(n-6,9,13)-trienoic acid, arachidonic acid, γ-linolenic acid, timnodonic acid, hexaenoic acid and mixtures thereof.

Emollients are often incorporated into cosmetic compositions of the present invention. Levels of such emollients may range from about 0.5% to about 50%, preferably between about 5% and 30% by weight of the total cosmetic composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate.

Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eruct and stearyl oleate. Preferred esters include coco-caprylate/caprate(a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere from 0.1 to 20% by weight, preferably from about 0.5% to 10% by weight of the total composition. Exemplary thickeners are cross-linked polyacrylate materials available under the trademark Carbopol from the B. F. Goodrich Company. Nonionic cellulose materials such as methyl cellulose and hydroxy propyl methyl and cellulose may be used. Also cationic cellulose materials such as polymer JR400 and cationic gums such as Jaguar 135 may be used as thickeners.

Surfactants, which are also sometimes designated as emulsifiers, may be incorporated into the cosmetic compositions of the present invention. Surfactants can comprise anywhere from about 0.5 to about 30%, preferably from about 1 to about 15% by weight of the total composition. Surfactants may be cationic, nonionic, anionic, or amphoteric in nature and combinations thereof may be employed.

The composition according to the invention can also contain other optional adjuncts, that is ingredients other than the main ingredients already defined which are conventionally employed in compositions for topical application to human skin. These adjuncts, when present, will normally form the balance of the composition.

Examples of optional adjuncts include vehicles, the selection of which will depend on the required product form of the composition. Typically, the vehicle when present, will be chosen from diluents, dispersants or carriers for the ingredients so as to ensure an even distribution of it when applied to the skin.

The compositions may include water as a vehicle in combination with at least one other cosmetically-acceptable vehicle.

Vehicles other than water that can be used in compositions according to the invention can include liquids or solids as emollients, solvents, humectants, thickeners and powders. Examples of each of these types of vehicles, which can be used singly or as mixtures of one or more vehicles, are as follows:

Emollients, such as stearyl alcohol, glyceryl monolaurate, glyceryl monoricinoleate, glyceryl monostearate, propane-1,2-diol, butane-1,3-diol, docosan-1,2-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, , isopropyl stearate, butyl stearate, polyethylene glycol, triethylene glycol, lanolin, cocoa butter, corn oil, cotton seed oil, tallow, lard, olive oil, palm kernel oil, rapeseed oil, safflower seed oil, soybean oil, sunflower seed oil, olive oil, sesame seed oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate;

Propellants, such as trichlorofluoromethane, dichlorodifluoromethane, dichlorotetrafluoroethane, monochlorodifluoromethane, trichlorotrifluoroethane, propane, butane, isobutane, dimethyl ether, carbon dioxide, nitrous oxide;

Solvents, such as ethyl alcohol, methylene chloride, isopropanol, acetone, castor oil, ethylene glycol monoethyl ether, diethylene glycol monobutyl ether, diethylene glycol monoethyl ether, dimethyl sulphoxide, dimethyl formamide, tetrahydrofuran;

Humectants, such as glycerin, sorbitol, sodium 2-pyrrolidone-5-carboxylate, soluble collagen, dibutyl phthalate, gelatin;

Powders, such as chalk, talc, fullers earth, kaolin, starch, gums, colloidal silicondioxide, sodium polyacrylate, tetra alkyl and/or trialkyl aryl ammonium smectites, chemically modified magnesium aluminum silicate, organically modified montmorillonite clay, hydrate aluminum silicate, fumed silica, carboxyvinyl polymer, sodium carboxymethyl cellulose, ethylene glycol monostearate.

The cosmetically acceptable vehicle, when present, will usually form from 0.01 to 99.9%, preferably from 50 to 98% by weight of the composition, and can, in the absence of other cosmetic adjuncts, form the balance of the composition.

The composition according to the invention can take the form of a liquid or gel, intended to be dispensed from a capped container such as a bottle, roll-on applicator or tube, or a pump-operated or propellant driven aerosol dispenser, as a skin cleanser, shower product, bath additive or shampoo. The composition can also take the form of a powder or a solid such as a stick, preferably housed in a suitable capped holder with a wind-up or push-up action similar to a lip stick, or a bar or tablet, with or without fatty acid soaps, intended to be used for washing instead of a conventional soap bar.

Compositions of the invention may be formulated as products for washing the skin, for example, bath or shower gels, hand washing compositions or facial washing liquids; pre- and post-shaving products; rinse-off, wipe-off and leave-on skin care products; and products for washing the hair.

The liquid compositions of the invention will generally be pourable liquids or semi-liquids e.g., pastes and will have a viscosity in the range 250 to 100,000 mPas measured at a shear rate 10s⁻¹ and 25° C., in a Haake Rotoviscometer RV20.

The invention also provides a closed container containing a detergent composition as herein defined.

In order to determine the efficacy of the compositions of the invention against prickly heat, it must be remembered that the condition is believed to be brought about by blockage of sweat glands (e.g., either by bacterial interference at too alkaline pH or by irritation when pH is too acidic). Thus, as will be seen in the methodologies aid examples, different ways of measuring the effect of compositions are:

(1) to measure the number of unblocked glands or ducts (more unblocked means less redness and irritation) when using composition of one pH relative to another; and

(2) to visually measure number of prickly heat outbreaks.

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

The following examples are intended to further illustrate the invention and are not intended to limit the invention in any way.

Unless indicated otherwise, all percentages are intended to be percentages by weight.

Methodology

1. Determining Blockage

One method of measuring prickly heat is to determine how many sweat glands are unobstructed via the so-called “starch/iodine” procedure.

According to this procedure, a 3% solution of potassium iodide and iodine is prepared in 95% ethanol and applied to the skin. After drying, a small volume of cornstarch in castor oil is spread over the iodine-treated skin. The preferred conditions for inducing sweating are a temperature of 40° C. and a relative humidity of 40%. As the subject sweats, the water droplets interact with the iodine and starch to form purple dots, representing functioning sweat glands, on the skin. The greater the number of dots, the more functioning (unblocked) glands. The number of purple dots can be imaged using a variety of techniques such as a digital camera (with or without a dermatoscope), video microscopy, or imprints onto bibulous or cigarette papers. All images can be counted either manually or by image analysis.

In the studies reported a Nikon Cool Pix 990 digital camera was employed in conjunction with a Heine Dermatoscope Delta 10. The images were counted manually from the computer screen.

2. Inducing Blockage and Effect of Pretreatment

In connection with this sweat gland measurement, it is first necessary to induce sweat gland blockage and to see what the effect of various pretreatments is. A brief discussion of these follows:

Occlusion Procedure Four Inducing Blockage

Prolonged exposure of the skin to sweat increases its hydration, and the first event in the production of prickly heat may be an increase in the resident skin flora. It is believed that occlusion increases skin hydration, which in turn results in significant increases in the level of bacteria on the skin. Occlusion of the skin is easily obtained by use of impermeable plastic, for example, Saran™ Wrap. Both Hill Top Chambers® (Hill Top Research, Inc.) and Tegaderm™ dressing (3M) with a Saran™ Wrap patch function equally well at providing an impermeable seal and are less cumbersome to use than plastic wrap alone. The largest Hill Top Chamber® is ˜4.9 cm2 while Tegaderm™ can be purchased in sizes of 19.4 cm² or 42 cm².

The skin is occluded for 72 to 96 hours. In order to avoid hydration effects, the chambers are always removed approximately three hours prior to sweat assessment.

Effect of Pre-Treatments

In order to study the ability of various pre-treatments to prevent or alleviate prickly heat, products were applied to the skin prior to placement of the occlusive material noted above. For use with Hill Top Chambers®), 49 mg or μl of product was applied to the skin in an area the size of the chamber. Liquids were contained within a plastic ring and allowed to air dry, while creams/lotions were spread with a spatula and then rubbed into the skin with a gloved finger. After treatment application, the skin was occluded and then assessed for unblocked sweat glands using method described in section one.

Optimization of the Starch/Iodine Procedure

After many evaluations, it was determined that the iodine solution was applied most uniformly with a Kimwipe rolled around a glass tube and the starch/oil mixture was best spread with a glass rod as described above. Both paper imprinting methods (bibulous and cigarette) were cumbersome, and the dots quickly became smeared and difficult to count. Digital camera images, alone or in conjunction with a Dermatoscope, were clear, easy to count, and could be stored on a CD. The only disadvantage with the Dermatoscope was the smaller area imaged. Although the video microscopy option also is quite suitable, it is not as readily accessible as a digital camera, and due to the small area visualized, multiple locations need to be imaged.

Sweat assessments are difficult to obtain immediately after the occlusive material is removed. The significantly hydrated skin obscures the purple dots by forming a diffuse purple area wherever the iodine and starch are applied. As a result, it was found that sweat assessments were best performed at least 3 hours after removal of the chamber.

Optimization of the Occlusion Procedure

It was found that 72-hour occlusion resulted in significant blockage of the eccrine glands and a subsequently was used for the majority of testing.

Both the lower forearms and upper back are suitable locations for occlusion. It should be noted, however, that there can be large differences in the number of functioning sweat glands at different locations on the body.

Various formulations discussed in the subsequent examples are set forth below.

TABLE 1

| | | | | Low pH | |
|---|---|---|---|---|---|
| | Syndet Bar | Syndet | | Syndet Bar | Acidic o/w |
| Ingredients (% by wt.) | (Market) | Liquid | Soap Base | (Prototype) | Emulsion |
| Sodium Soap | 5-12 | | 75-90 | | |
| Sodium Cocoyl | 30-45 | | | 35-50 | |
| Isethionate | | | | | |
| Cocamidopropyl Betaine | 0.5-5 | 2-8 | | 0.5-5 | |
| Palmitic/Stearic Acid | 15-25 | | | 10-16 | 2-8 |
| Coconut Fatty Acid | 1-5 | | | 2-8 | |
| Sodium Isethionate | 1-5 | | | 1-5 | |
| Sodium Stearate | 1-5 | | | 1-5 | |
| Sodium Lauryl Ether | | 7-20 | | | |
| Sulfate | | | | | |
| Glycerin | | 2-20 | | | 1-10 |
| PEG-40 Hydrogenated | | 0.1-1 | | | |
| Castor Oil | | | | | |
| Hydroxypropyl Guar | | 0.01-1 | | | |
| Hydroxypropyltrimonium | | | | | |
| Chloride | | | | | |
| Propylene Glycol | | | | 8-20 | |
| Butylene Glycol | | | | | 1-7 |
| Succinic Acid | | | | 1-6 | |
| Hydroxycaprylic Acid | | | | | 0.05-2 |
| Glycolic Acid | | | | | 5-10 |
| Vegetable Oil | | | | 2-10 | |
| Glyceryl | | | | | 0.5-3.5 |
| Hydroxystearate | | | | | |
| Stearyl Alcohol | | | | | 0.5-3.5 |
| Isostearyl Palmitate | | | | | 5-15 |
| Sorbitan monostearate | | | | | 0.5-4 |
| PEG-100 Stearate | | | | | 1-6 |
| Sodium Stearoyl | | | | | 0.4-2.5 |
| Lactylate | | | | | |
| Dimethicone | | | | | 0.1-2 |
| Calcium Carbonate | 2-10 | | | | |
| Magnesium Aluminum | | | | | 0.2-1.4 |
| Silicate | | | | | |
| Hydroxyethylcellulose | | | | | 0.1-1.2 |
| Xanthan Gum | | | | | 0.1-0.5 |
| Perfume, Color, | 0.5-5 | 0.5-5 | 0.5-5 | 0.5-5 | 0.5-5 |
| Stabilizer, Preservatives | | | | | |
| Water | To balance | To balance | To balance | To balance | To balance |
| pH | 7.0-7.5 | 5.0-5.5 | 8.5-9.5 | 4.0-5.5 | 3.8 |

### EXAMPLE 1

### Effect of Pre-treatments on Number of Unblocked Pores

In order to show the effect of compositions of various product pH on prickly heat, applicants tested the effect of various compositions on pore blockage, which is directly correlated to prickly heat condition (i.e., greater number of blocked pores equals susceptibility to prickly heat while, conversely, more unblocked pores equals less susceptibility). Pretreatment studies in this regard are set forth below.

In the pre-treatment studies, Hill Top Chambers® (small, impermeable plastic chamber used to produce an occlusive environment designed, for purposes of the invention, to mimic prickly heat condition). These typically were used due to their small size, and during the initial evaluations, 48 or 72-hour occlusion as described in Methodology Section was employed. Several very small-scale studies were performed to determine how different materials would affect eccrine gland blockage under occlusion. Measurement of pore blockage is as set forth in methodology section. The findings are summarized below.

Study 1: Pore Blockage Based on Acidic o/w Emulsion Versus Occluded Untreated Site

A low pH (acidic) oil-in-water emulsion was compared to an occluded, untreated site on the upper back. Both formulations were tested using Hill-Top® Chamber. The results are shown in FIG. 1. The skin pH increased over time (e.g., from 5.4 to 5.9) for the occluded site and remained at a low pH (e.g., 4.1) for the emulsion. In addition, the number of unblocked sweat glands was higher at each time point for the emulsion (e.g., 440 v. 199 at 24 hr. and 331 v. 78 at 48 hours). The results thus clearly indicate that the low pH emulsion product (pH 4.1) helped prevent eccrine gland blockage as compared to occlusion alone. Typically, occlusion produces an alkaline pH environment very similar to the conditions of massive sweating and the use of alkaline cleansers therefore, the finding is consistent with the belief that higher product pH (too alkaline) will lead to an environment where bacteria will more readily flourish and create more blocked glands.

Study 2: Pore Blockage Effect Based on Lanolin, Isoproponol, Various Buffers and Occluded and Unoccluded Controls

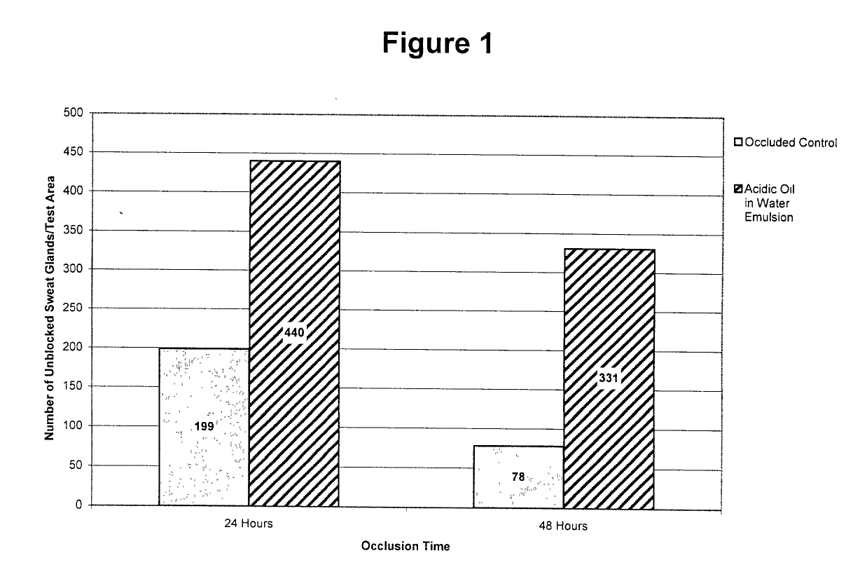

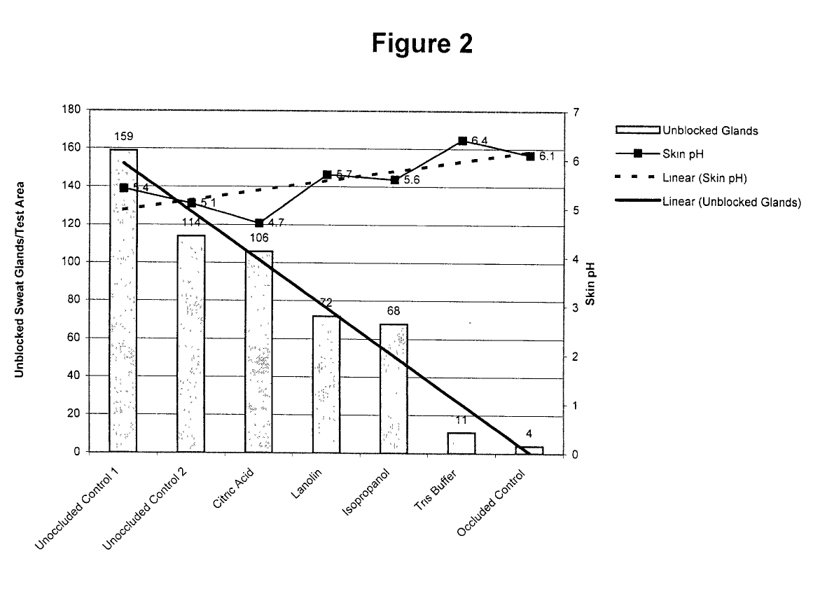

The following different materials were evaluated in this test: lanolin, 70% isopropanol, and 0.1M citric acid and Tris buffers, as well as occluded and unoccluded control sites. The results are presented in FIG. 2. Compared to the occluded control site (pH of 6.1), the citric acid buffer (pH of 4.7), lanolin (pH of 5.7) and isopropanol (pH of 5.6) reduced the number of blocked sweat glands, while the Tris buffer had no positive effect. In general, it appears that as the skin pH approaches 6 and up under occlusion, the number of unblocked sweat glands decreases. Without wishing to be bound by theory, this may be because the more acidic site, while less mild, has greater antibacterial effect. It is not, however so acidic that the “irritation” effect begins to set in where obstruction would again be seen. Further, as pH goes too high (e.g., Tris Buffer), there is much more blockage (e.g., due to growth of bacteria).

### EXAMPLE 2

### Effect of Bar Products

Study 1

Solutions of plain soap (without antibacterial agents) (Table 1, column 3) and a low pH 4 syndet prototype bar (containing succinic acid) (Table 1, column 4) were compared to unoccluded and occluded control sites after 72-hours occlusion on the forearm. In addition, the number of organisms present after occlusion also were determined.

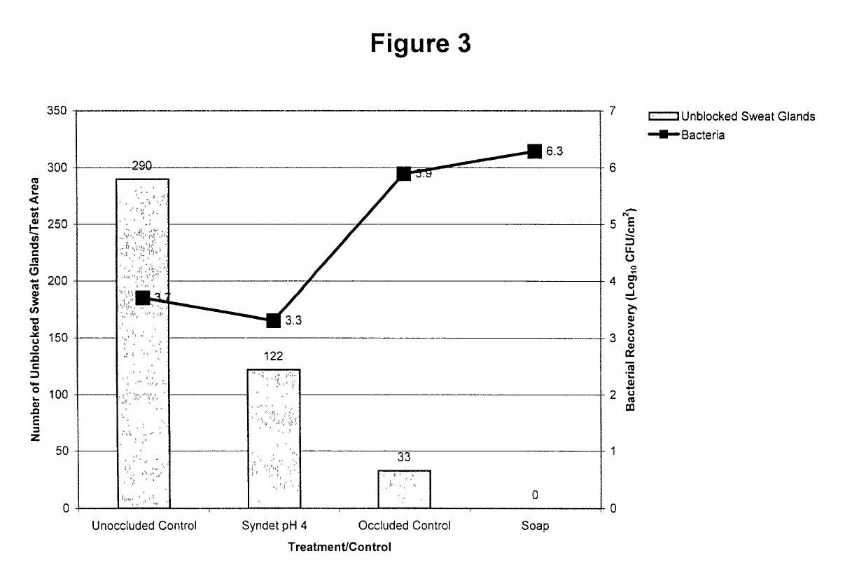

The numbers of unblocked sweat glands and bacteria for the four sites after 72-hours occlusion are shown in FIG. 3. Compared to the unoccluded control, the syndet site had a reduced amount of unblocked sweat glands, and, despite the occlusive environment, a slightly lower number of bacteria. Compared to these two sites, both the occluded control and alkaline soap (pH 8.5) sites had significant reductions in the number of unblocked glands (more occlusion) and much higher levels of bacteria.

The study again shows the balance between sufficiently acid to fight bacterial and stop blockage (without being too acidic and causing irritation) and, on the other hand, not so alkaline as to allow bacteria to flourish and cause damage.

Study 2

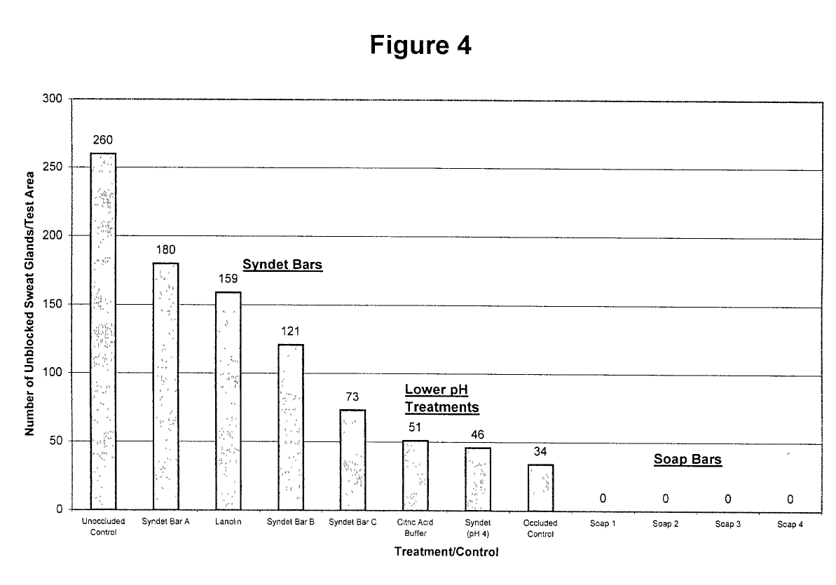

Several different soap and syndet bar solutions were evaluated on the back of one subject. Both lanolin and the citric acid buffer were included as benchmarks. The results are presented in FIG. 4.

The overall trend was that all soaps (all being alkaline with pH 8.5 and greater) completely blocked all sweat glands in the area treated. Syndet bars, however, kept significant number of sweat glands unobstructed. The more neutral syndets caused the least blockage. The skin pH was obtained from all sites and, as expected, the highest pH readings (most alkaline) were from the sites treated with the soap solutions.

### EXAMPLE 3

To show effect of pH clinically, applicants conducted the following test.

Experimental

Overall Study Design

A 24-week, double-blind, randomized and balanced monadic study was conducted consisting of 5 cells. A sufficient number of subjects were screened to finish with approximately 100 subjects per cell. Indonesian male children of ages 8-12 (inclusive) who met the prescreening criteria were required to visit for a screening visit. An evaluator visually assessed the subjects to determine if they met all the inclusion/exclusion criteria and if any pre-existing skin conditions would preclude them from entering the study.

Qualified subjects were balanced into one of five cells and entered into the product application phase (Week 0). One cell of subjects continued using their current products with their normal wash regimen (referred to as Control Group). The Control Group was able to use any product of choice including cooling powder and Calamine® lotion. The balance of subjects were required to use the provided text products twice a day for the duration of the study. All subjects were required to make at least six more visits to the test center over a 24-week period of time (at Weeks 4, 8, 12, 16, 20, and 24). Prickly heat assessments and skin pH measurements were conducted during all test center visits. In addition, home visits to subjects were made at least twice between test center visits. Visual assessment, self-assessment questionnaires, and compliance were conducted during the home visits.

Results

The products used in the five cells were:

TABLE 2

| | Product pH |
|---|---|
| Cell 1: (n = 110) Syndet Bar (Market) | 7.3 |
| Cell 2: Syndet Liquid (n = 108) (Market) | 5.0 |
| Cell 3: Plain Soap Bar (n = 112) | 8.5 |
| Cell 4: Low pH Syndet Bar (n = 109) | 5.5 |
| Cell 5: Control Group (n = 110) | Range depending |
| | on product chosen |

The formulations are set forth below in Table 1, previously given.

The results of observation are set forth below in Tables 3

| | |
|---|---|
| Cell 1: Syndet Bar (Market) | Cell 2: Syndet Liquid |
| Cell 3: Soap Base | Cell 4: Low pH Syndet (pH 5.5) |
| Cell 5: Control Group | |

TABLE 3
Summary of Prickly Heat Outbreaks During Study

| Summary Statistics | Cell 1 | Cell 2 | Cell 3 | Cell 4 | Cell 5 |
|---|---|---|---|---|---|
| Total # of Subjects | 101 | 102 | 101 | 101 | 100 |
| Total # of Prickly Heat | 23 | 31 | 43 | 47 | 38 |
| outbreaks | | | | | |
| Average Days to | 5.17 | 4.65 | 6.19 | 4.77 | 4.66 |
| Resolution of Prickly | | | | | |
| Heat Outbreak | | | | | |

Except for Cell 4, the results are consistent with the invention: both syndet products caused less prickly heat outbreaks than the soap did. Also, the average number of days to resolution of given outbreaks was lower for the syndet group compared to the soap group. It is believed that Cell 4 is an artifact caused by the fact that the bar (due to insufficient hardness) was not rinsed properly from the skin and therefore itself was causing occlusion.

### EXAMPLE 4

A study was conducted to determine the effect of pH on EPS production.

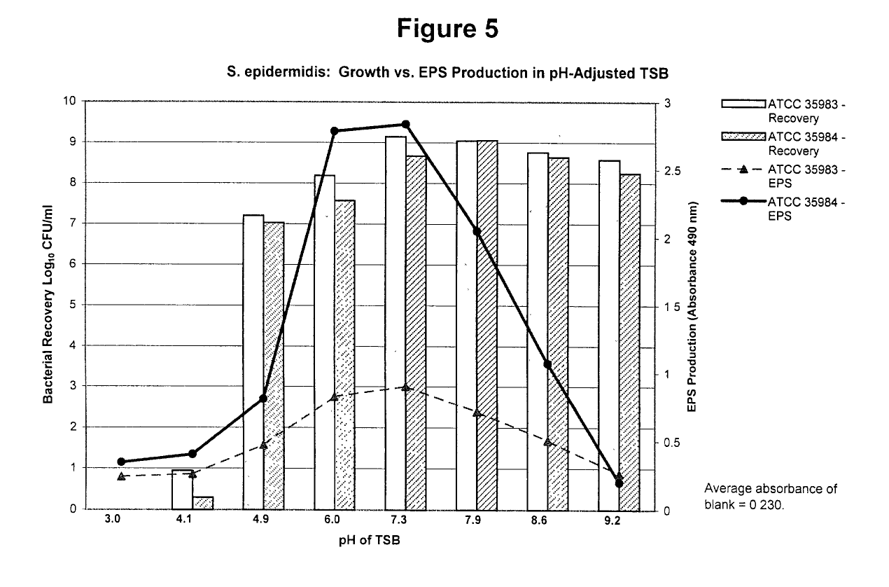

As seen in FIG. 5, optimal EPS production (which causes obstruction and is, therefore, bad) occurs at pH above 6.5 to 7.5. This is the pH which results when a conventional alkaline soap is added to normally acidic skin.

### EXAMLE 5

A study was conducted to investigate the effects of various alkaline and non-alkaline products on skin pH.

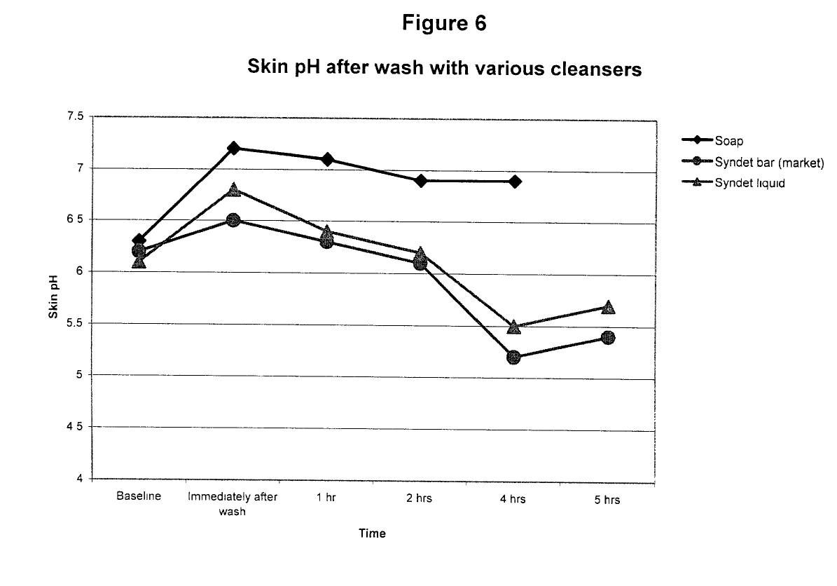

As seen from FIG. 6, non-soap products (syndet liquid and syndet bar) maintain more acidic skin pH (but above 4) compared to soap where skin pH is near 7. This in turn is optimum area for EPS production which EPS causes more blockage of glands and leads to prickly heat. The non soaps have lower pH, will not form an environment optimal for EPS and therefor will not enhance obstruction.

### EXAMPLE 6

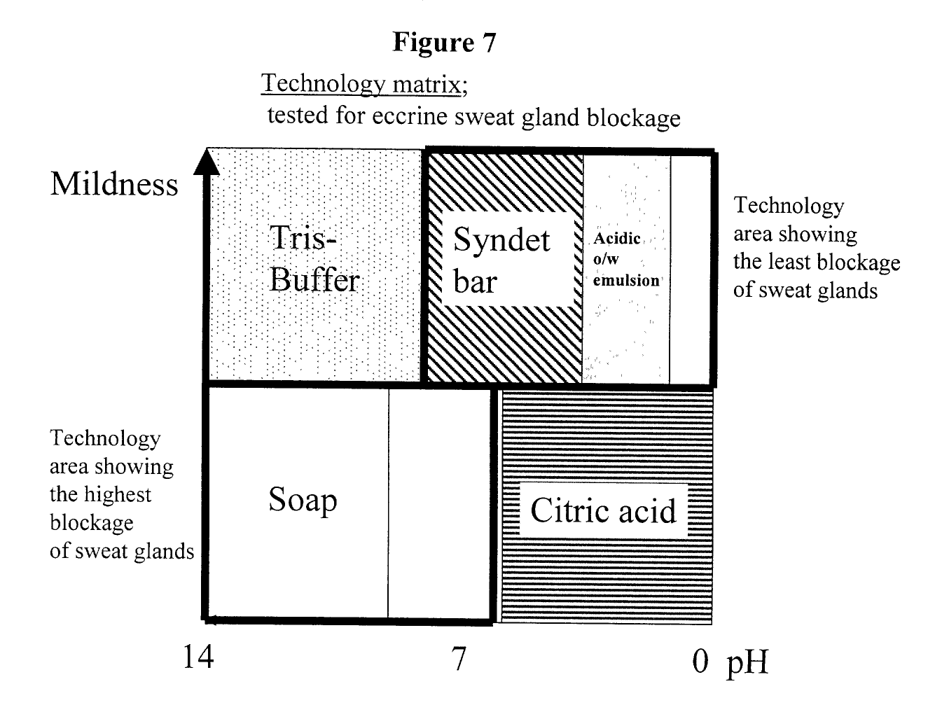

In order to observe overall pH and mildness effects on gland blockage, applicants prepared a grid showing the various effects. As seen from FIG. 7, the least blockage occurs when there is combination of both lower pH and mildness (upper right) while greatest blockage occurs when there is combination of higher pH and less mildness (lower pH).

1. A method of preventing and/or alleviating the effects of prickly heat by applying to skin a personal wash cleanser product having pH of greater than about 4.0 to less than about 7.5

2. A method according to claim 1, wherein the cleanser form is selected from the group consisting of bar, liquid, cream and emulsion in a form suitable for application to skin.

3. A method for increasing number of unblocked pore sites by applying to skin a personal wash cleanser having a pH greater than about 4.0 to less than 7.5
